(19) United States
(12) Patent Application Publication
(10) Pub. No.: US 2015/0272449 A1
(43) Pub. Date: Oct. 1, 2015

(54) Hybrid Intravascular Pressure Measurement Devices and Associated Systems and Methods

(71) Applicant: Volcano Corporation, San Diego, CA (US)

(72) Inventor: Douglas E. Meyer, Folsom, CA (US)

(21) Appl. No.: 14/663,123

(22) Filed: Mar. 19, 2015

Related U.S. Application Data
(60) Provisional application No. 61/970,771, filed on Mar. 26, 2014.

(51) Int. Cl. A61B 5/0215; A61B 19/00; A61B 5/00; A61M 25/09

(52) U.S. Cl. CPC A61B 5/0215; A61M 25/09; A61B 19/54; A61B 5/742; A61B 2019/5466; A61M 2210/12; A61M 2230/30

(57) ABSTRACT

Intravascular devices, systems, and methods are disclosed. In some embodiments, an intravascular pressure measurement device is provided. The intravascular device includes a flexible elongate member with a proximal portion and a distal portion and a lumen extending therethrough. The lumen is configured to allow the passage of a guidewire. The distal portion of the member includes first and second distal sections. The second distal section having an outer diameter that is smaller than the outer diameter of the first distal section. The intravascular device further includes a first pressure sensor disposed within the wall of the first distal section of the flexible elongate member to measure the pressure within the lumen.

### CROSS REFERENCE TO RELATED APPLICATIONS

The present application claims priority to and the benefit of the U.S. Provisional Patent Application No. 61/970,771, filed Mar. 26, 2014, which is hereby incorporated by reference in their entirety

### TECHNICAL FIELD

Embodiments of the present disclosure relate generally to the field of medical devices and, more particularly, to a device, system, and method for measuring pressure within vessels. Aspects of the present disclosure are particularly suited for evaluation of a lesion within a human blood vessel.

### BACKGROUND

Heart disease is a serious health condition affecting millions of people worldwide. One major cause of heart disease is the presence of blockages or lesions within the blood vessels that reduce blood flow through the vessels.

Improved techniques for assessing the functional significance and likely benefit of treatment of a stenosis in a blood vessel are the calculation of fractional flow reserve (FFR). FFR is defined as the ratio of the maximal hyperemic blood flow in a stenotic artery compared to what the maximal flow would be if the stenosis were alleviated. FFR provides an index of stenosis severity that allow determination if the obstruction limits blood flow within the vessel to an extent that intervention is warranted, taking into consideration both the risks and benefits of treatment. The more restrictive the stenosis, the greater the pressure drop across the stenosis, and the lower the resulting FFR or instantaneous wave-free ratio.

One method for measuring the proximal and distal pressures used for FFR calculation is to advance a pressure sensing guidewire (with a pressure sensor embedded near its distal tip) across the lesion to a distal location, while the guiding catheter (with an attached pressure transducer or fluid column) is used to provide a pressure measurement proximal to the stenosis (typically in the aorta or the ostium of the coronary artery). Despite the level of evidence in the guidelines, the use of pressure sensing guide wires remains relatively low (estimated less than 6% of cases worldwide). The reasons are partially tied to the performance of the pressure guide wires relative to that of standard angioplasty wires. Incorporating a pressure sensor into a guidewire generally requires compromises in the mechanical performance of the guidewire in terms of steerability, durability, stiffness profile, etc., that make it more difficult to navigate the coronary circulation to deliver the guidewire or subsequent interventional catheters across the lesion. As such, physicians will often abandon use of a pressure sensing guidewire when they experience challenges steering the pressure guide wire distal to the disease.

Another method of measuring the pressure gradient across a lesion is to use a small catheter connected to an external blood pressure transducer to measure the pressure at the tip of the catheter through a fluid column within the catheter, similar to the aortic catheter pressure measurement. However, this method can introduce error into the FFR calculation because as the catheter crosses the lesion, it creates additional obstruction to blood flow across the stenosis and contributes to a lower distal blood pressure measurement than what would be caused by the lesion alone, exaggerating the apparent functional significance of the lesion. Additionally, the size of the catheter may complicate the collection of pressure measurement data.

While the existing treatments have been generally adequate for their intended purposes, they have not been entirely satisfactory in all respects. The devices, systems, and associated methods of the present disclosure overcome one or more of the shortcomings of the prior art.

### SUMMARY

In one embodiment, an intravascular pressure measurement device is provided. The intravascular device includes a flexible elongate member with a proximal portion and a distal portion and a lumen extending therethrough. The lumen is sized and shaped to allow the passage of a guidewire therethrough. The distal portion of the member includes a first distal section and a second distal section. The first distal section has a first outer diameter and an opening at a distal end thereof. The second distal section has a second outer diameter that is smaller than the first outer diameter, with a proximal end of the second distal section being coupled to a distal end of the first distal section. The intravascular device further includes a first pressure sensor disposed within the wall of the first distal section of the flexible elongate member, such that the pressure sensor has access to measure the pressure within the lumen.

In another embodiment, a system for obtaining intravascular measurements is provided. The system includes a processing system that has a processor in communication with a memory and an acquisition module and also includes an intravascular device. The intravascular device includes a flexible elongate member with a proximal portion and a distal portion and a lumen extending therethrough that is sized and shaped to allow the passage of a guidewire. The distal portion of the member has a first distal section with a first outer diameter and an opening at a distal end thereof. The second distal section has a second outer diameter that is smaller than the first outer diameter, a proximal end of the second distal section being coupled to a distal end of the first distal section. The distal end of the second distal section being a distal end of the flexible elongate member. Additionally, the intravascular device includes a pressure sensor disposed within the wall of the first distal section of the flexible elongate member. The pressure sensor is configured to have access to the lumen and is coupled to the acquisition module to obtain pressure measurement data.

In yet another embodiment, a method of measuring pressure within a vessel lumen having a lesion therein is provided. The method includes steps of positioning a guidewire within the vessel lumen proximate the lesion and advancing an intravascular pressure measurement device over the guidewire such that a distal end of the intravascular pressure measurement device is positioned adjacent to the lesion. The intravascular pressure measurement device has a first distal section having a first outer diameter, the first distal section coupled to a second distal section having a second outer diameter that is smaller than the first outer diameter. The method also includes a step of withdrawing the guidewire from at least a portion of a lumen of the intravascular pressure measurement device to expose a pressure sensor to the lumen. The lumen has a pressure related to a pressure at the distal end of the intravascular pressure measurement device. The method further includes a step of obtaining pressure measurement data using the pressure sensor.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is diagram of a medical system according to some embodiments of the present disclosure.

FIG. 2A is a side-view diagram of an intravascular device for use in the medical system of FIG. 1 according to embodiments of the present disclosure.

FIG. 2B is a cross-sectional side-view diagram of the intravascular device as presented in FIG. 2A according to embodiments of the present disclosure.

FIG. 3A is a close-up of a portion of the cross-sectional side-view diagram of the intravascular device according to embodiments of the present disclosure.

FIG. 3B is a close-up of a portion of the cross-sectional side-view diagram of an alternative intravascular device according to embodiments of the present disclosure.

FIGS. 4A, 4B, and 4C are plots of actual or measured pressure levels within a vessel of a patient according to some embodiments of the present disclosure.

FIG. 5 is a flowchart of a method of measuring pressure within a vessel having a lesion therein according to embodiments of the present disclosure.

For clarity of discussion, elements having the same designation in the drawings may have the same or similar functions. The drawings may be better understood by referring to the following Detailed Description.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. For the sake of brevity, however, the numerous iterations of these combinations will not be described separately. For simplicity, in some instances the same reference numbers are used throughout the drawings to refer to the same or like parts.

The present disclosure relates generally to devices, systems, and methods of using a pressure-sensing intravascular device or catheter, in some embodiments, for the assessment of intravascular pressure, including, by way of non-limiting example, the calculation of an FFR value or other pressure ratio calculation. These measurements can be made in the coronary vessels. These measurements can also be made in the peripheral vasculature including but not limited to the superficial femoral artery (SFA), below the knee (BTK, i.e. tibial), and Iliac artery. In some instances, embodiments of the present disclosure are configured to measure the pressure proximal to and distal to a stenotic lesion within a blood vessel. Embodiments of the present disclosure include a pressure sensor embedded in the wall of the intravascular device. In some embodiments, the pressure-sensing catheter disclosed herein includes at least one perfusion port extending through the catheter wall to allow for blood flow through the catheter lumen. In some embodiments, the pressure-sensing intravascular device disclosed herein is configured as a rapid exchange catheter. In other embodiments, the pressure-sensing intravascular device disclosed herein is configured as a conventional over-the-wire catheter. The pressure-sensing intravascular devices disclosed herein enable the user to obtain pressure measurements using an existing guidewire, such as a conventional 0.014 inch guidewire, that can remain fairly stationary through the pressure measurement procedure. Thus, the pressure-sensing intravascular devices disclosed herein enable the user to obtain physiologic information about an intravascular lesion without losing the original position of the guidewire. Embodiments of the present disclosure further include a distal portion of the intravascular device that exhibits more than one outer diameter, such that the impact of the intravascular device on the pressure within the vessel being measured is minimized.

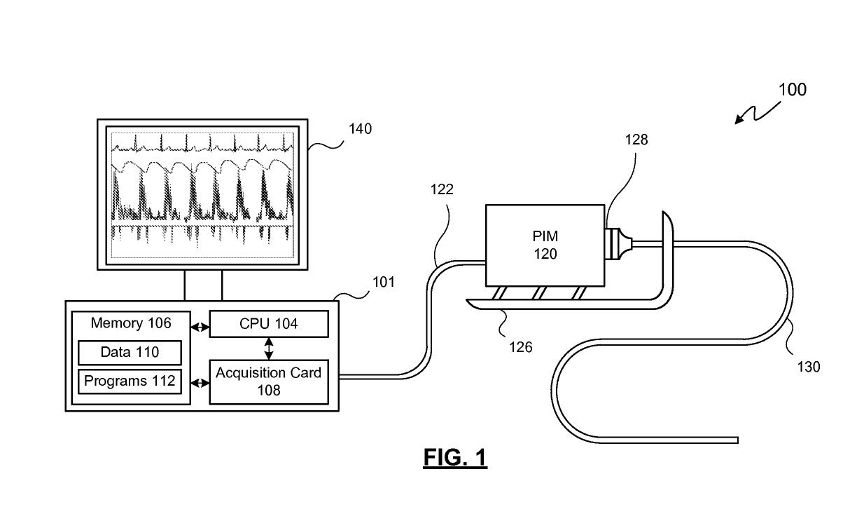

Referring to FIG. 1, shown therein is a medical system 100 for collecting and processing pressure measurement data that shares a number of features with the system 100 described above in connection with FIG. 1. The system 100 includes a controller 102, which may be a workstation-type controller or may be a hand-held computing device such as a tablet computing device. The controller 102 includes one or more processors, illustrated as central processing unit (CPU) 104, in communication with a memory 106 and a data acquisition card 108, which may include a plurality of analog and digital components and field programmable gate arrays. The memory 106 may include multiple types of memory and/or multiple levels of memory. Thus, memory 106 may include random access memory (RAM), read only memory (ROM), a hard disk drive, a solid-state drive, etc. The memory 106 stores data 110, which may include pressure measure data obtained using an intravascular device configured for pressure measurement, parameter settings therefor, and programs 112, which may provide for pressure measure data collection, for the manipulation and processing of collected data, and for the selection and implementation of settings and parameters associated with the pressure measurement collection process and related devices.

The acquisition card 108 provides an interface between the controller 102 and a PIM 120, being coupled thereto by a link 122. In some embodiments, more than one acquisition card may be present in the system 100. For example, a first acquisition card 108 may be present on the controller 102, while another is present in the PIM 120 or in another controller such as a bedside box. The PIM 120 may include a sled 126 that can be used to move the PIM 120 and thereby the intravascular device 130 during controlled translational movements, such as a “pullback” movement. The PIM 120 includes a device socket 128 that is used to couple the intravascular device 130 to the PIM 120. When in use, data obtained using the intravascular device 130 may be displayed to a monitor 140, which may also be used to display imaging data obtained using another intravascular device or imaging components configured within the intravascular device 130.

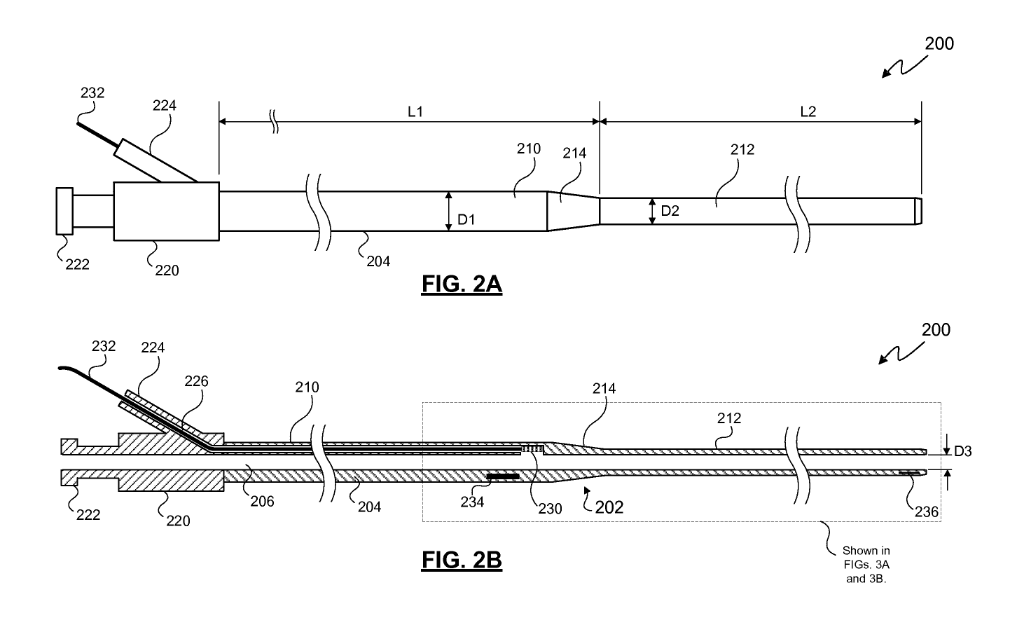

FIG. 2A illustrates a side view of an intravascular device 200, which may be used in embodiments of the system 100 as the intravascular device 130 of FIG. 1. Thus, the intravascular device 200 is an intravascular pressure measurement device. The intravascular device 200 is configured to measure pressure within a tubular structure (e.g., a blood vessel) according to one embodiment of the present disclosure. In some embodiments, the intravascular device 200 is used in the medical system 100 to calculate a pressure ratio (i.e. FFR) based on the obtained pressure measurements. The intravascular device 200 includes a flexible elongate member 202. The flexible elongate member 202 comprises a wall 204 that defines an internal lumen 206 (seen in the cross-section of FIG. 2B). In general, the flexible elongate member 202 is sized and shaped for use within an internal structure of a patient, including but not limited to a patient's arteries, veins, heart chambers, neurovascular structures, gastrointestinal system, pulmonary system, and/or other areas where internal access of patient anatomy is desirable. In the pictured embodiment, the flexible elongate member 202 is shaped and sized for intravascular placement.

In particular, the flexible elongate member 202 is shaped and configured for insertion into a lumen of a blood vessel such that a longitudinal axis of the intravascular device aligns with a longitudinal axis of the vessel at any given position within the vessel lumen. In that regard, the straight configuration illustrated in FIG. 2 is for exemplary purposes only and in no way limits the manner in which the intravascular device 200 may curve, bend, torque, pivot, or otherwise change orientations in use. Generally, the flexible elongate member 202 may be configured to take on any desired arcuate profile(s) necessary to advance through a vessel. The flexible elongate member 202 is formed of a flexible material such as, by way of non-limiting example, plastics, high density polyethylene, polytetrafluoroethylene (PTFE), Nylon, block copolymers of polyamide and polyether (e.g., PEBAX), thermoplastic, polyimide, silicone, elastomers, metals, shape memory alloys, polyolefin, polyether-ester copolymer, polyurethane, polyvinyl chloride, combinations thereof, or any other suitable material for the manufacture of flexible, elongate intravascular devices such as catheters.

The flexible elongate member 202 has a combined length labeled in FIG. 2 as the sum of a length L1 and a length L2. The length L1 may range from about 100 centimeters to about 300 centimeters, and define a first portion of the flexible elongate member 202, only a distal portion 210 of which is shown in FIG. 2. This first distal section 210 has an outer diameter D1, which may range from about 0.026 inches to about 0.053 inches. The distal end of the first distal section 210 of the flexible elongate member 202 is integrally formed with and/or coupled to a proximal end of a second distal section 212. The second distal section 212 is shorter than the first distal section 210 and has the length L2, which may range from about 10 centimeters to about 30 centimeters. As seen in FIGS. 2A and 2B, the second distal section 212 may be coupled to the first distal section 210 by a coupling section 214 that tapers from the outer diameter D1 of the first distal section 210 to an outer diameter D2 of the second distal section 212. The outer diameter D2 may range from about 0.007 inches to about 0.033 inches.

Because of the smaller outer diameter D2 of the second distal section 212, the distal section 212 may be fabricated in a separate process from a process used to create the first distal section 210. For example, the second distal section 212 may be formed by an additive process in which layer upon layer of material is formed over a cylindrical substrate, which is then removed. After being formed separately, the first distal section 210 and the second distal section 212 may be joined together by overmolding, thermoforming, and/or another appropriate coupling process.

As seen in FIGS. 2A and 2B, the intravascular device 200 also includes an adapter 220 coupled to a proximal end of the flexible elongate member 202. The adapter 220 has a connector 222 through which a guidewire may be passed. Additionally, the adapter 220 includes an access port 224. The access port 224 provides access to an ancillary lumen 226 (seen in FIG. 2B) that extends through a portion of the adapter 220 and partially through the first distal section 210 of the flexible elongate member 202.

Referring now to FIG. 2B, a cross-sectional view of the intravascular device 200 is illustrated therein. The cross-sectional view of FIG. 2B provides a clearer view of the lumens in the intravascular device 200. The lumen 206 extends through the connector 222, which may include a Luer-type connector at its proximal end and through the first distal section 210 and the second distal section 212 of the flexible elongate member 202. As illustrated, the lumen 206 extends along a central axis of the flexible elongate member 202 and has an inner diameter D3, which is sufficiently large to accommodate a 0.014 inch guidewire therethrough. In the illustrated embodiment, D3 is constant along the length of the lumen 206. In other embodiments, D3 varies along the length of the lumen 206. For example, in some embodiments a distal section of the lumen 206 has a diameter and/or cross-sectional area that is smaller than a proximal section of the lumen 206. A second lumen 226 extends through the access port 224 and the wall 204 of the first distal section 210. The lumen 226 extends to a distal portion of the first distal section 210. At a distal end of the lumen 226, there is a chamber 228 that is configured to accommodate a pressure sensor 230.

As illustrated, the pressure sensor 230 is a piezoelectric sensor, such as a piezoresistive sensor. However, in other embodiments, the pressure sensor 230 may be a capacitive pressure sensor, a fiber optic pressure sensor, or a fluid-column pressure sensor. The pressure sensor 230 is configured in the distal end of the lumen 226 such that it measures the pressure within the lumen 206. Thus, the pressure sensor 230 has access to the lumen 206. In some instances, a diaphragm of the pressure sensor 230 is exposed to the lumen 206. The pressure sensor 230 is coupled to a controller, such as the PIM 120 of FIG. 1 for the transmission of pressure measurement data obtained using the pressure sensor 230. The pressure sensor 230 may be coupled to the PIM 120 by the communication cable 232 that extends through the lumen 226. Communication cable 232 may include electrical, optical, and/or other communication lines.

As described herein, the outer diameter of D2 of the second distal portion 212 is smaller than the outer diameter D1 of the first distal portion 210. The smaller diameter D2 decreases the impact on the pressure within the vessel lumen of the vessel in which measurements are obtained. To obtain pressure measurements using the pressure sensor 230 of the intravascular device 200, the lumen 206 is filled with a saline solution prior to positioning within the vessel of a patient. The fluid, i.e. blood, of the patient fluidly communicates with the fluid filling the lumen 206 such that a pressure exerted at the distal end of the second distal section 212 is also exerted along the lumen 206, including at the distal end of the first distal section 210, which includes the pressure sensor 230.

To facilitate the desired placement of the flexible elongate member 202 within the vessel of the patient, the intravascular device 200 includes at least one radiopaque marker 234. Some embodiments also include a radiopaque marker 236 disposed at the distal end of the second distal section 212. Each radiopaque marker present in the flexible elongate member 202 may be coupled to or positioned within the wall 204 of the flexible elongate member 202 at a known distance from the pressure sensor 230 and/or the distal end of the second distal portion 212. The radiopaque markers 234 and/or 236 permit a physician to fluoroscopically visualize the location and orientation of the markers, the distal end of the second distal portion 212, and the pressure sensor 230 within the patient. For example, when the second distal portion 212 extends into a blood vessel in the vicinity of a lesion, X-ray imaging of the radiopaque markers 234 and/or 236 may confirm successful positioning of the pressure sensor 230 distal to or proximal to the lesion. In some embodiments, the radiopaque markers 234 and/or 236 may circumferentially surround the flexible elongate member 202. In other embodiments, the radiopaque markers 234 and/or 236 may be shaped and configured in any of a variety of suitable shapes, including, by way of non-limiting example, rectangular, triangular, ovoid, linear, and non-circumferential shapes. The radiopaque markers 234 and 236 may be formed of any of a variety of biocompatible radiopaque materials that are sufficiently visible under fluoroscopy to assist in the procedure. Such radiopaque materials may be fabricated from, by way of non-limiting example, platinum, gold, silver, platinum/iridium alloy, and tungsten. The markers 234 and 236 may be attached to the catheter 100 using a variety of known methods such as adhesive bonding, lamination between two layers of polymers, or vapor deposition, for example. Various embodiments may include any number and arrangement of radiopaque markers. In some embodiments, the intravascular device 200 lacks radiopaque markers.

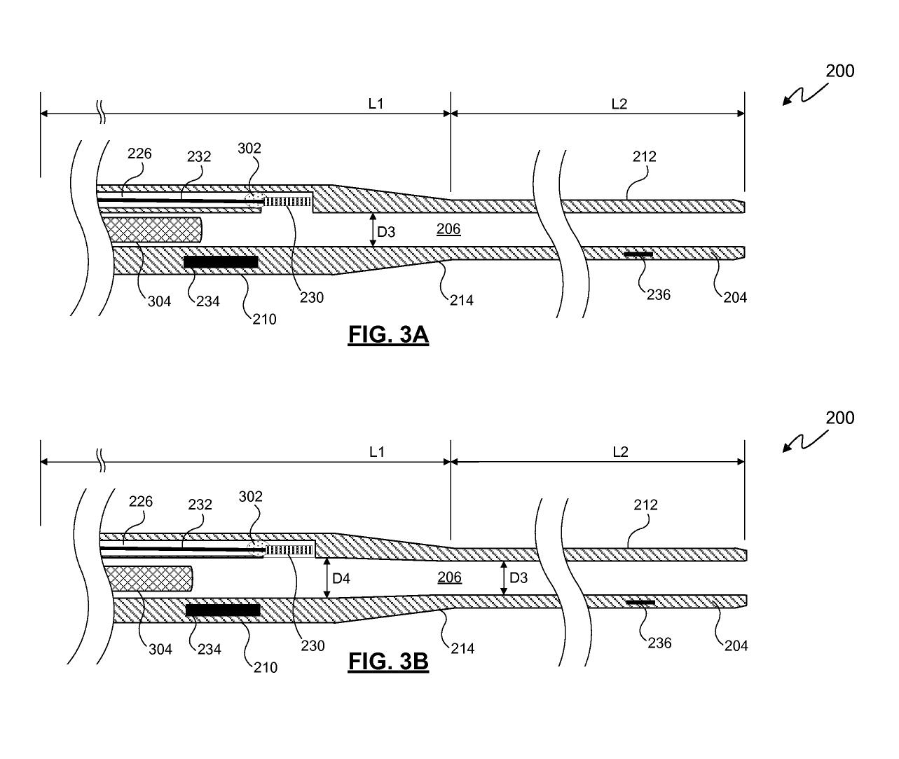

Referring now to FIG. 3A, a close-up view of a portion of the flexible elongate member 202 is shown therein. As illustrated in FIG. 3A, the ancillary lumen 226 that contains the communication cable 232 and the pressure sensor 230 also includes a sealant or adhesive 302. The adhesive 302 both secures the pressure sensor 230 in position within the chamber at the distal end of the ancillary lumen 226 and prevents fluid from exiting the lumen 206 through the ancillary lumen 226. In some embodiments, the adhesive 302 secures the pressure sensor 230 in position, but does not seal off of the ancillary lumen 226. In such embodiments, a fluid may be injected into the lumen 206 through the ancillary lumen 226 prior to positioning the flexible elongate member 202 within a vessel of a patient.

As illustrated in FIG. 3A, a guidewire 304 is positioned within the lumen 206. The guidewire 304 has been withdrawn from beyond a distal end of the flexible elongate member 202 to a position within the lumen 206 that is proximal to the pressure sensor 230. The guidewire 304 is used to facilitate the desired positioning of the flexible elongate member 202. By subsequently withdrawing the guidewire 304 beyond the pressure sensor 230, the pressure within the lumen 206 more closely approximates the pressure present at the distal end of the second distal section 212 (i.e., the distal most tip of the intravascular device 200), thereby increasing the accuracy of pressure measurement data obtained using the pressure sensor 230.

Referring now to FIG. 3B, the embodiment of the flexible elongate member 202 illustrated therein includes an inner diameter of the lumen 206 that changes along the length thereof. As illustrated the second distal section 212 has the inner diameter D3, while the first distal section 210 has an inner diameter D4. The inner diameter D4 is larger than the inner diameter D3. The coupling section 214 may include a tapered section of the lumen 206, such that the lumen 206 has the diameter D4 at a proximal end of the coupling section 214 and the diameter D3 at the distal end thereof. Accordingly, the coupling section 214 may include both internal (i.e., lumen) and external tapers.

For embodiments of the flexible elongate member 202 as seen in FIG. 3A and as seen in FIG. 3B, a pressure drop may occur between the distal end of the second distal section 212 and the location within the lumen 206 that is exposed to the pressure sensor 230. Thus, the pressure at the distal tip of the intravascular device 200 may be higher than at the pressure sensor 230 where pressure measurement data is obtained.

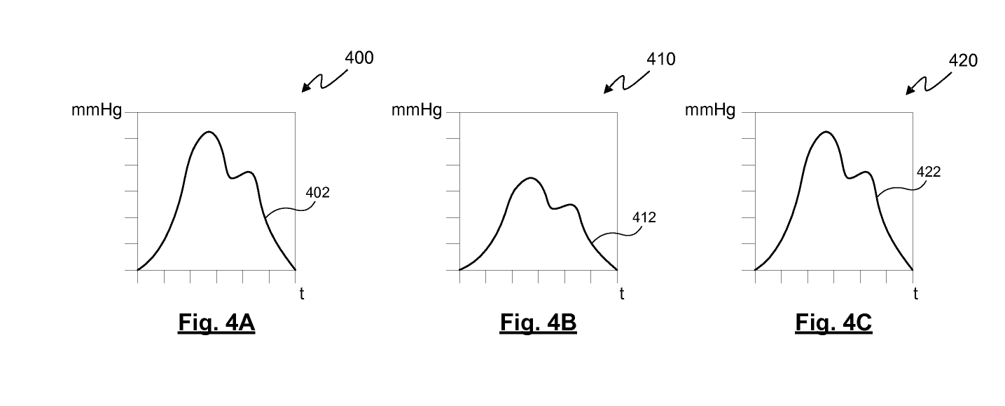

Referring now to FIGS. 4A-C, FIG. 4A illustrates exemplary pressure 402 plotted with time on the x-axis and pressure in mmHg on the y-axis in a chart 400. The chart 400 illustrates an example of pressure present at the distal tip of the intravascular device 200. FIG. 4B illustrates exemplary pressure data 412 in a chart 410. The exemplary pressure data 412 represents pressure measurement data corresponding to the exemplary pressure 402 of FIG. 4A, but obtained using the pressure sensor 230 proximal of the distal tip of the intravascular device 200. In an example, the pressure present at the distal tip of the intravascular device 200 as seen in chart 400 may have a mean value of around 100 mmHg, while the exemplary pressure data 412 has a mean value of around 45 mmHg. As such, the pressure measurement data obtained using the pressure sensor 230 may be compensated according to a calibrated factor to correct the data. Thus, the exemplary pressure data 412 may be communicated over the wires 232 to the PIM 120 and/or the controller 102 of FIG. 1, where the compensation is applied to generate compensated pressure data 422 as seen in the chart 420 of FIG. 4C that more closely or exactly matches the pressure data 402 in chart 400. It is understood that linear, non-linear, polynomial, and/or compensation factors may be utilized. In some instances, intravascular device 200 is calibrated relative to known pressure(s) to determine the appropriate calibration factor(s) for the intravascular device 200.

The pressure measurement data obtained using the intravascular device 200 may be combined with data obtained from another sensor positioned to collected data on an opposite side of the lesion. The use of two pressure measurement data sets may allow the determination of the impact of the lesion on pressure and/or flow within neighboring sections of the vessel being observed, such as by FFR. Having a clear indication of the impact of the lesion permits a doctor overseeing treatment of the patient to make better-informed treatment decisions, which often leads to improved outcomes for the patient. In some instances, pressure ratio calculations are performed as disclosed in U.S. patent application Ser. No. 13/420,296, filed on Apr. 30, 2012, which is hereby incorporated by reference in its entirety.

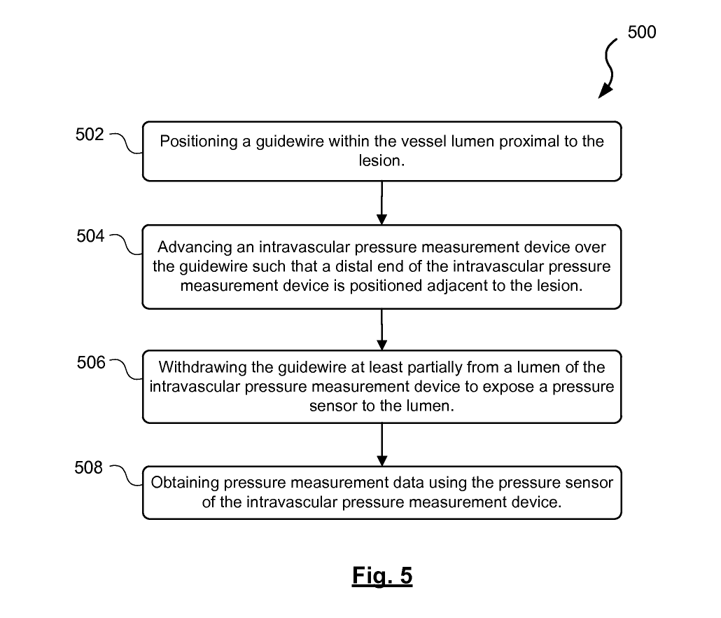

FIG. 5 is a flowchart of a method 500 of measuring pressure within a vessel lumen having a lesion therein. As illustrated, the method 500 includes several enumerated steps. However, embodiments of the method 500 may include additional steps, before, after, in between, and/or as part of the enumerated steps. Thus, as illustrated the method 500 begins in step 502 in which a surgeon positions a guidewire within the vessel lumen adjacent to the lesion. The guidewire may be the guidewire 304 illustrated in FIGS. 3A and 3B. In step 504, an intravascular pressure measurement device is advanced over the guidewire such that a distal end of the intravascular pressure measurement device is positioned adjacent to the lesion. In that regard, the distal end can be positioned distal of the lesion and/or proximal of the lesion. For example, in some instances the distal end is positioned distal of the lesion and then pulled back to a position proximal of the lesion. Similarly, in some instances the distal end is positioned proximal of the lesion and then pushed through to a position distal of the lesion. The intravascular device has a first distal section with a first outer diameter. The first distal section is coupled to a second distal section that has a second outer diameter smaller than the first outer diameter. For example, the intravascular pressure measurement device may be this intravascular device 200 as illustrated in FIGS. 2A, 2B, 3A, and 3B and having the features described herein.

In step 506, the surgeon withdraws the guidewire at least partially from a lumen of the intravascular pressure measurement device to expose a pressure sensor to the lumen. For example, the guidewire 304 may be withdrawn or retracted as seen in FIGS. 3A and 3B, such that the distal end of the guidewire 304 is positioned proximal of the pressure sensor 230, but remains within the lumen 206. In other embodiments, the guidewire 304 may be completely withdrawn and the lumen 206 may be sealed to prevent fluid from exiting through it. In step 508, the pressure sensor is used to obtained pressure measurement data. The pressure measurement data obtained in step 508 may be combined with pressure measurement data obtained using another pressure sensor to calculated FFR values associated with the lesion.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example the intravascular devices described herein may be utilized anywhere with a patient's body, including both arterial and venous vessels, having an indication for pressure measurement. It is understood that such variations may be made to the foregoing without departing from the scope of the present disclosure. Accordingly, the following claims should be construed broadly and in a manner consistent with the present disclosure.

What is claimed is:

1. An intravascular pressure measurement device comprising:
a flexible elongate member with a proximal portion and a distal portion, the flexible elongate member having a lumen extending therethrough, the lumen sized and shaped to allow the passage of a guidewire therethrough, wherein the distal portion of the member comprises:
a first distal section having a first outer diameter; and
a second distal section having a second outer diameter that is smaller than the first outer diameter, a proximal end of the second distal section being coupled to a distal end of the first distal section; and
a pressure sensor disposed within the wall of the first distal section of the flexible elongate member, the pressure sensor configured to measure a pressure within the lumen.

2. The intravascular pressure measurement device of claim 1, wherein the distal portion of the flexible elongate member further comprises a coupling section that couples the proximal end of the second distal section to the distal end of the first distal section.

3. The intravascular pressure measurement device of claim 2, wherein the coupling section is tapered such that the coupling section has a distal end outer diameter and a proximal end outer diameter, the distal end outer diameter being equal to the second outer diameter and the proximal end outer diameter being equal to the first outer diameter.

4. The intravascular pressure measurement device of claim 1, further comprising a luer-type connector at a proximal end of the flexible elongate member.

5. The intravascular pressure measurement device of claim 1, wherein the second outer diameter ranges from about 0.007 inches to about 0.033 inches and the first outer diameter ranges from about 0.026 inches to about 0.053 inches.

6. The intravascular pressure measurement device of claim 1, further comprising a radiopaque marker positioned within the second distal section.

7. The intravascular pressure measurement device of claim 6, further comprising a radiopaque marker positioned within the first distal section.

8. The intravascular pressure measurement device of claim 1, further comprising an ancillary lumen extending partway through the flexible elongate member, and wherein the first pressure sensor is positioned in a distal portion of the ancillary lumen.

9. The intravascular pressure measurement device of claim 1, wherein a diaphragm of the pressure sensor is exposed to the lumen.

10. The intravascular pressure measurement device of claim 1, wherein the second distal section has a length from about 10 centimeters to about 30 centimeters.

11. A system for obtaining intravascular measurements, the system comprising:
a processing system having a processor in communication with a memory and an acquisition module; and
an intravascular device in communication with the processing system, the intravascular device comprising:
a flexible elongate member with a proximal portion and a distal portion, the flexible elongate member having a lumen extending therethrough, the lumen sized and shaped to allow the passage of a guidewire therethrough, wherein the distal portion of the member comprises:
a first distal section having a first outer diameter; and
a second distal section having a second outer diameter that is smaller than the first outer diameter, a proximal end of the second distal section being coupled to a distal end of the first distal section; and
a pressure sensor disposed within the wall of the first distal section of the flexible elongate member, the pressure sensor configured to measure a pressure within the lumen.

12. The system of claim 11, further comprising a display in communication with the processing system to display obtained pressure measurement data.

13. The system of claim 11, wherein the pressure sensor is configured to obtain absolute pressure measurement data.

14. The system of claim 11, wherein the second outer diameter ranges from about 0.007 inches to about 0.033 inches and a length of the second distal section ranges from about 10 to centimeters to about 30 centimeters.

15. The system of claim 11, further comprising a guidewire configured to be positioned at least partially within the lumen.

16. The system of claim 11, wherein the pressure sensor is a piezoresistive pressure sensor.

17. The system of claim 11, wherein the first distal section has a first lumen diameter that is larger than a second lumen diameter of the second distal section.

18. A method of measuring pressure within a vessel lumen having a lesion therein, the method comprising:
positioning a guidewire within the vessel lumen proximate the lesion;
advancing an intravascular pressure measurement device over the guidewire such that a distal end of the intravascular pressure measurement device is positioned adjacent to the lesion, the intravascular pressure measurement device having a first distal section having a first outer diameter and a second distal section having a second outer diameter that is smaller than the first outer diameter;
withdrawing the guidewire at least partially from a lumen of the intravascular pressure measurement device to a position proximal of a pressure sensor of the intravascular pressure measurement device, the lumen having a pressure related to a pressure at the distal end of the intravascular pressure measurement device; and
obtaining pressure measurement data using the pressure sensor.

19. The method of claim 18, wherein the pressure measurement data is obtained using the pressure sensor with the distal end of the intravascular pressure measurement device at a position distal of the lesion.

20. The method of claim 19, further comprising:
obtaining additional pressure measurement data proximal of the lesion;
calculating a pressure ratio based on the pressure measurement data obtained distal of the lesion and proximal of the lesion; and
displaying the calculated pressure ratio to a user.
